(19) United States
(12) Patent Application Publication
(10) Pub. No.: US 2025/0050056 A1
(43) Pub. Date: Feb. 13, 2025

(54) SYSTEM FOR PROVIDING SLEEP HEALTHCARE SERVICE USING ARTIFICIAL INTELLIGENCE-BASED BRAINWAVE ENTRAINMENT AND AUTONOMIC NERVOUS SYSTEM CONTROL

(71) Applicants: JangWon LEE, Seoul (KR); Meta Therapeutics Inc., Gwangju (KR)

(72) Inventor: JangWon LEE, Seoul (KR)

(73) Assignees: JangWon LEE, Seoul (KR); Meta Therapeutics Inc., Gwangju (KR)

(21) Appl. No.: 18/796,420

(22) Filed: Aug. 7, 2024

(30) Foreign Application Priority Data
Aug. 8, 2023 (KR) 10-2023-0103492

(51) Int. Cl. A61M 21/02; A61B 5/00; A61N 1/36

(52) U.S. Cl. CPC A61M 21/02; A61B 5/4806; A61N 1/36025

(57) ABSTRACT

Disclosed is a system for providing a sleep healthcare service using brainwave entrainment and autonomous nervous system control, and the system including: a user terminal configured to set a bedtime and a wake-up time, at the bedtime, output a sleep-inducing breathing method for enhancing the parasympathetic nervous system, while emitting light of non-melatonin-suppressing wavelengths, and at the wake-up time, insert and output an Alpha-Band or Beta-Band flicker for brainwave synchronization of alpha or beta waves, and output blue light to induce arousal; and a sleep healthcare server.

### BACKGROUND OF THE INVENTION

The present disclosure relates to a system for providing a sleep healthcare service using brainwave entrainment and autonomic nervous system control and, more specifically, to a system that normalizes sleep and circadian rhythms by performing brainwave entrainment and autonomic nervous system control through light, sound, breathing, and vibrations.

As society progresses, sleep disorders such as insomnia arise due to various stress factors. The Korean Standard Classification of Diseases (KCD) defines sleep disorders under the disease code G47. Subcategories of G47 include insomnia-related diseases (G47.0) and sleep-wake disorders (G47.2). Adequate sleep restores health and vitality and is closely related to various human hormones. Therefore, when proper sleep is not achieved, one may experience fatigue and lethargy daily. In severe cases, one can be exposed to serious illnesses such as chronic fatigue, dementia, depression, and panic disorder. As the rate of sleep disorders increases among modern people, the necessity of sleep care also rises.

Methods have been researched and developed to help people fall asleep using monaural beats to create brainwave pulsations or sound and light therapy. Related technologies include Korean Patent Application Publication No. 2023-0080260 (published on Jun. 7, 2023) and Korean Patent Application Publication No. 2012-0131253 (published on Dec. 5, 2012), which disclose configurations for mixing monaural beats in waveforms to induce brainwave entrainment, loading frequency data according to reference frequencies for brainwave entrainment, adjusting decibels, and generating superimposed monaural beats as wave files to provide to user terminals, as well as configurations for detecting sleep states using piezoelectric sensors and providing light and sound therapy.

However, the former only discloses a method for mixing monaural beats without providing configurations for regulating brainwaves and the autonomic nervous system according to sleep and wake cycles. The latter also only discloses light and sound therapy without configurations utilizing monaural or binaural beats, and the type of light therapy is not specified. Brainwaves and the autonomic nervous system are controlled according to sleep and wake patterns. In cases of insomnia or hypersomnia, there is a tendency for brainwaves and autonomic nervous system control to deviate from normal ranges. In addition, abnormal activation in the hypothalamus, which controls circadian rhythms, may disrupt these rhythms and cause cluster headaches, depression, ADHD, anxiety disorders, obsessive-compulsive disorder, panic disorder, and other symptoms. Therefore, research and development of a system to ultimately restore circadian rhythms by managing sleep cycles are required.

### SUMMARY OF THE INVENTION

In view of the above, the present disclosure provides a system for providing a sleep healthcare service using brain entrainment and autonomic nervous system control, the system achieving brainwave entrainment using light and sound and controlling the autonomic nervous system according to a bedtime and a wake-up time, playing monaural or binaural beats through a speaker at the wake-up time to induce theta, alpha, and beta brainwaves, offering a sunlight shower with blue light at the wake-up time to enhance arousal and promote the secretion of serotonin, facilitating serotonin conversion into melatonin at the bedtime to promote sleep, manually or in response to a monitored stress level exceeding a threshold, providing bilateral alternating stimulation in tactile form through a user terminal and a wearable device to reduce stress, and alleviating pain using non-invasive vagus nerve stimulation while alleviating cluster headaches caused by hypothalamic-pituitary axis hyperactivity through normalization of circadian rhythms. However, the objectives of the present disclosure are not limited to the aforementioned objective, and other not-mentioned objectives may be included.

In one general aspect, there is provided a system for providing a sleep healthcare service using brainwave entrainment and autonomous nervous system control, and the system includes: a user terminal configured to set a bedtime and a wake-up time, at the bedtime, output a sleep-inducing breathing method for enhancing the parasympathetic nervous system, while emitting light of non-melatonin-suppressing wavelengths, and at the wake-up time, insert and output an Alpha-Band or Beta-Band flicker for brainwave entrainment of alpha or beta waves, and output blue light to induce arousal; and a sleep healthcare server comprising a database for storing settings related to light, sound, vibration, and a breathing method for brainwave entrainment and autonomic nervous system control at the bedtime and the wake-up time, a setting part for receiving bedtime and the wake-up time settings from the user terminal, and a controller for outputting at least one of the light, sound, vibration, and breathing method for brainwave entrainment and autonomic nervous system control at the bedtime and the wake-up time set by the user terminal.

According to the present disclosure, it is possible to achieve brainwave entrainment using light and sound and control the autonomic nervous system according to a bedtime and a wake-up time; play monaural or binaural beats through a speaker at a wake-up time to induce theta, alpha, and beta brainwaves; offer a sunlight shower with blue light at the wake-up time to enhance arousal and promote the secretion of serotonin; facilitate the conversion of serotonin into melatonin at the bedtime to promote sleep; provide bilateral alternating stimulation in tactile form through a user terminal and a wearable device to reduce stress manually or in response to a monitored stress level exceeding a threshold; and alleviate pain using non-invasive vagus nerve stimulation while normalizing circadian rhythms to alleviate cluster headaches caused by hypothalamic-pituitary axis hyperactivity.

### BRIEF DESCRIPTION OF THE DRAWINGS

FIG. 1 is a diagram illustrating a sleep healthcare service system using brainwave entrainment and autonomic nervous system control according to an embodiment of the present disclosure.

FIG. 2 is a block diagram illustrating a sleep healthcare server included in the system of FIG. 1.

FIGS. 3 and 4A-4N are diagrams for explaining examples in which a sleep healthcare service using brainwave entrainment and autonomic nervous system control according to an embodiment of the present disclosure is implemented.

FIG. 5 is an operation flowchart illustrating a method of providing a sleep healthcare service using brainwave entrainment and autonomic nervous system control according to an embodiment of the present disclosure.

### DETAILED DESCRIPTION OF THE INVENTION

Hereinafter, exemplary embodiments of the present disclosure will be described in detail with reference to the accompanying drawings. The present disclosure, however, may be modified in various different ways, and should not be construed as limited to the embodiments set forth herein. For clarity of the disclosure, irrelevant parts are removed from the drawings, and similar reference denotations are used to refer to similar elements throughout the specification.

In embodiments of the present disclosure, when an element is “connected” with another element, the element may be “directly connected” with the other element, or the element may be “electrically connected” with the other element via an intervening element. When an element “comprises” or “includes” another element, the element may further include, rather than exclude, the other element, and the terms “comprise” and “include” should be appreciated as not excluding the possibility of presence or adding one or more features, numbers, steps, operations, elements, parts, or combinations thereof.

When the measurement of an element is modified by the term “about” or “substantially,” if a production or material tolerance is provided for the element, the term “about” or “substantially” is used to indicate that the element has the same or a close value to the measurement and is used for a better understanding of the present disclosure or for preventing any unscrupulous infringement of the disclosure where the exact or absolute numbers are mentioned. As used herein, “step of” A or “step A-ing” does not necessarily mean that the step is one for A.

As used herein, the term “part” may mean a unit or device implemented in hardware, software, or a combination thereof. One unit may be implemented with two or more hardware devices or components, or two or more units may be implemented in a single hardware device or component. The “unit” is not limited to software or hardware devices or components and may advantageously be configured to reside on an addressable storage medium and configured to operate on one or more processors. Accordingly, the “unit” may include, for example, components, such as software components, object-oriented software components, class components, and task components, processes, functions, attributes, procedures, sub-routines, segments of program code, drivers, firmware, microcode, circuitry, data, databases, data structures, tables, arrays, and variables. The functionalities provided in the components and “units” may be combined into fewer components and “units” or may be further separated into additional components and “units.” Furthermore, the components and “units” may be implemented to operate on one or more CPUs within a device or a security multimedia card.

In the present specification, some of the operations or functions described as performed by a terminal, an apparatus, or a device may be performed instead in a server connected to the corresponding terminal, apparatus, or device. Similarly, some of the operations or functions described as being performed by a server may be performed in a terminal, an apparatus, or a device connected to the corresponding server.

In the present specification, some of the operations or functions described as mapping with or matching a terminal means mapping or matching a unique number of the terminal or personal identification information, which is identification data of the terminal.

Hereinafter, the present disclosure will be described in detail with reference to the attached drawings.

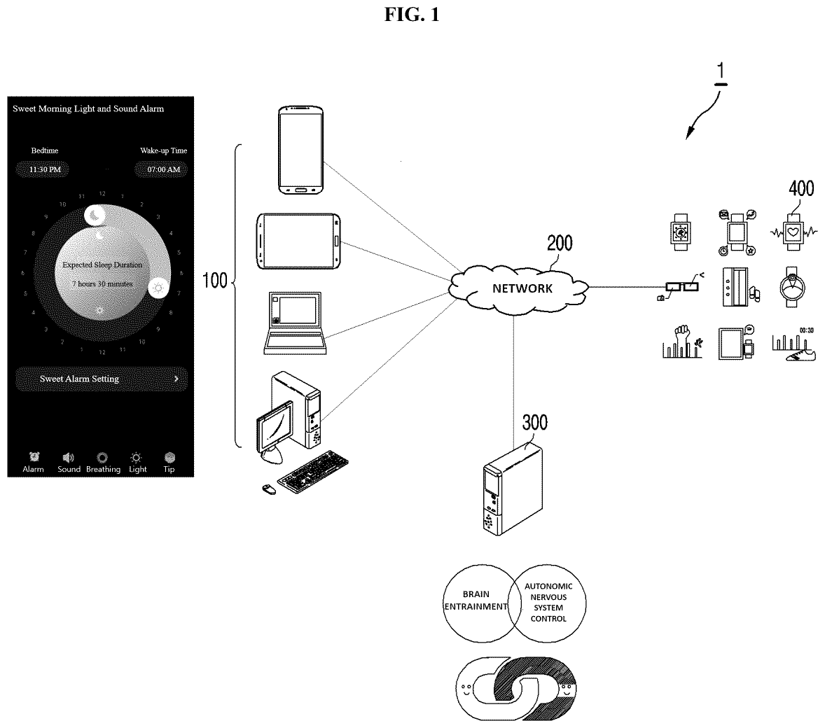

FIG. 1 is a diagram illustrating a sleep healthcare service system using brainwave entrainment and autonomic nervous system control according to an embodiment of the present disclosure. Referring to FIG. 1, a system 1 for providing a sleep healthcare service using brainwave entrainment and autonomic nervous system control may include at least one user terminal 100, a sleep healthcare server 300, and at least one wearable device 400. However, the system 1 for providing a sleep healthcare service using brainwave entrainment and autonomic nervous system control, as shown in FIG. 1, is merely an example of the present disclosure, and the present disclosure is not limited thereby.

In this case, each component of FIG. 1 is generally connected over a network 200. For example, as shown in FIG. 1, at least one user terminal 100 may be connected to the sleep healthcare server 300 over the network 200. In addition, the sleep healthcare server 300 may be connected to at least one user terminal 100 and at least one wearable device 400 over the network 200. Moreover, at least one wearable device 400 may be connected to the sleep healthcare server 300 over the network 200.

Here, the network refers to a connected structure that enables information exchange between nodes such as multiple terminals and servers. Examples of such a network include a Local Area Network (LAN), Wide Area Network (WAN), Internet (World Wide Web, WWW), wired and wireless data communication networks, telephone networks, and wired and wireless television communication networks. Examples of wireless data communication networks include 3G, 4G, 5G, 3rd Generation Partnership Project (3GPP), 5th Generation Partnership Project (5GPP), 5G New Radio (NR), 6th Generation of Cellular Networks (6G), Long Term Evolution (LTE), World Interoperability for Microwave Access (WiMAX), Wi-Fi, Internet, LAN, Wireless Local Area Network (Wireless LAN), WAN, Personal Area Network (PAN), Radio Frequency (RF), Bluetooth networks, Near-Field Communication (NFC) networks, satellite broadcast networks, analog broadcast networks, and Digital Multimedia Broadcasting (DMB) networks, but are not limited thereto.

In the following, the term “at least one” is defined as a term including the singular and plural, and even if the term “at least one” is not present, each component may be present in singular or plural, and it will be obvious that it may mean singular or plural. In addition, whether each component is provided in singular or plural forms can vary according to embodiments.

At least one user terminal 100 may be a user terminal that sets a bedtime and a wake-up time using a webpage, app page, program, or application related to a sleep healthcare service using brainwave entrainment and autonomic nervous system control, and outputs corresponding light, sound, vibration, and breathing method.

Here, at least one user terminal 100 may be implemented as a computer capable of accessing a server or terminal at a remote location over a network. The computer may include, for example, a navigation system, a laptop, a desktop, and a laptop equipped with a web browser (WEB Browser), etc. At least one user terminal 100 may be implemented as a terminal capable of accessing a server or terminal at a remote location over a network. At least one user terminal 100 may be a mobile communication device in which portability and mobility are guaranteed, and examples thereof may include all types of handheld-based wireless communication devices such as a personal communication system (PCS), global system for mobile communication (GSM), personal digital cellular (PDC), a personal handyphone system (PHS), a personal digital assistant (PDA), international mobile telecommunication (IMT)-2000, code division multiple access (CDMA)-2000, W-code division multiple access (W-CDMA), a wireless broadband Internet (WiBro) terminal, a smart phone, a smart pad, a tablet PC, and the like.

The sleep healthcare server 300 may be a server that provides web pages, app pages, programs, or applications related to the sleep healthcare service using brainwave entrainment and autonomic nervous system control. The sleep healthcare server 300 may also be a server that stores settings related to light, sound, vibration, and a breathing method in a database. In addition, the sleep healthcare server 300 may be a server that enables the output of at least one of light, sound, vibration, and breathing method for brainwave entrainment and autonomic nervous system control at the bedtime and wake-up time set by a user terminal 100. In addition, a sleep healthcare server 300 may be a server that provides Bilateral Alternating Stimulation in Tactile form (BLAST) through the user terminal 100 and a wearable device 400, and controls a vagus nerve stimulation device to alleviate pain through Non-Invasive Vagus Nerve Stimulation (nVNS) or outputs high-frequency signals to the user terminal 100.

Here, the sleep healthcare server 300 may be implemented as a computer capable of accessing a server or terminal at a remote location over a network. The computer may include, for example, a navigation system, a laptop, a desktop, and a laptop equipped with a web browser (WEB Browser), etc.

At least one wearable device 400 may be a device that uploads detection data to identify sleep stages using a sleep healthcare service related to brainwave entrainment and autonomic nervous system control through web pages, app pages, programs, or applications, and outputs vibrations of a preset frequency at a preset intensity and duration.

Here, at least one wearable device 400 may be implemented as a computer capable of accessing a server or terminal at a remote location over a network. The computer may include, for example, a navigation system, a laptop, a desktop, and a laptop equipped with a web browser (WEB Browser), etc. In this case, at least one wearable device 400 may be implemented as a terminal capable of accessing a server or terminal at a remote location over a network. At least one wearable device 400 may be a mobile communication device in which portability and mobility are guaranteed, and examples thereof may include all types of handheld-based wireless communication devices such as a personal communication system (PCS), global system for mobile communication (GSM), personal digital cellular (PDC), a personal handyphone system (PHS), a personal digital assistant (PDA), international mobile telecommunication (IMT)-2000, code division multiple access (CDMA)-2000, W-code division multiple access (W-CDMA), a wireless broadband Internet (WiBro) terminal, a smart phone, a smart pad, a tablet PC, and the like.

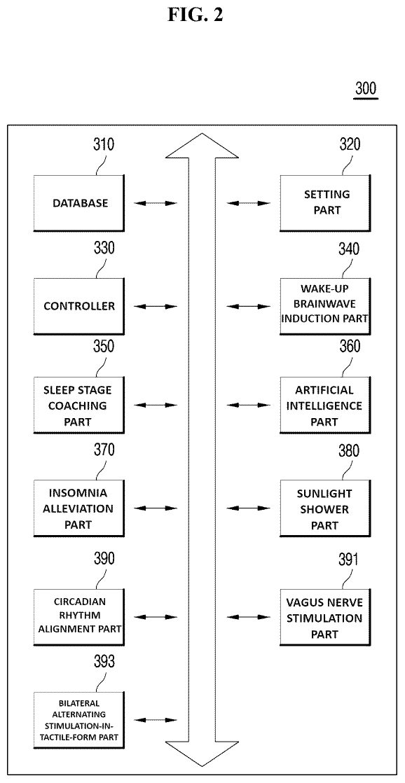

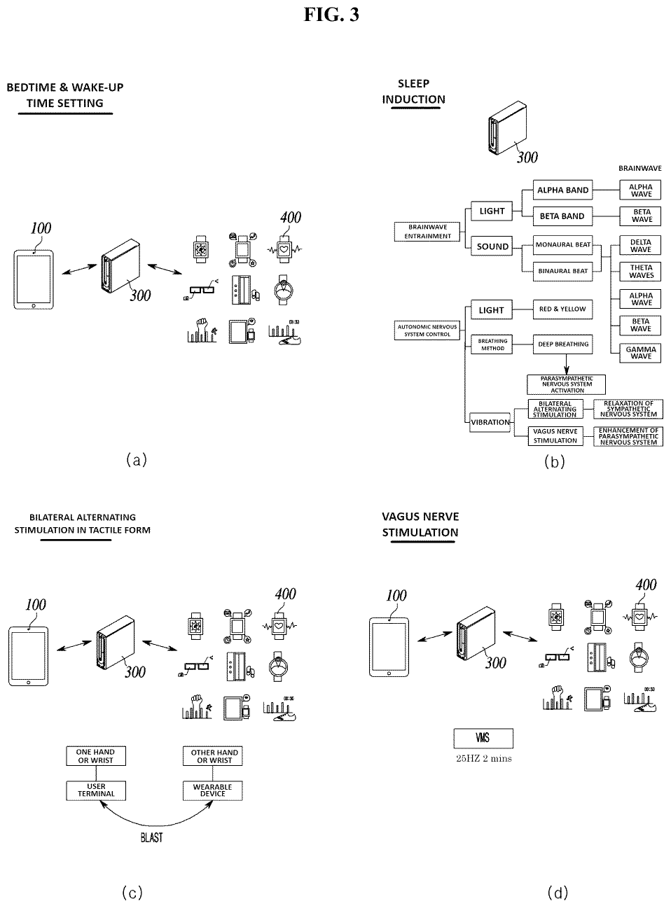

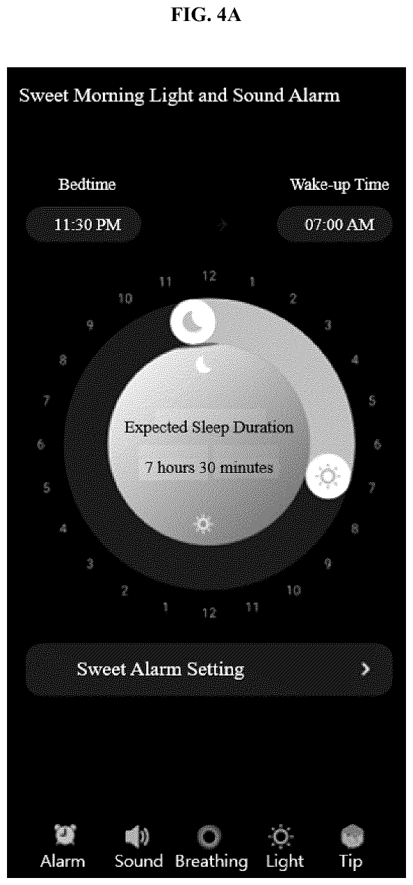

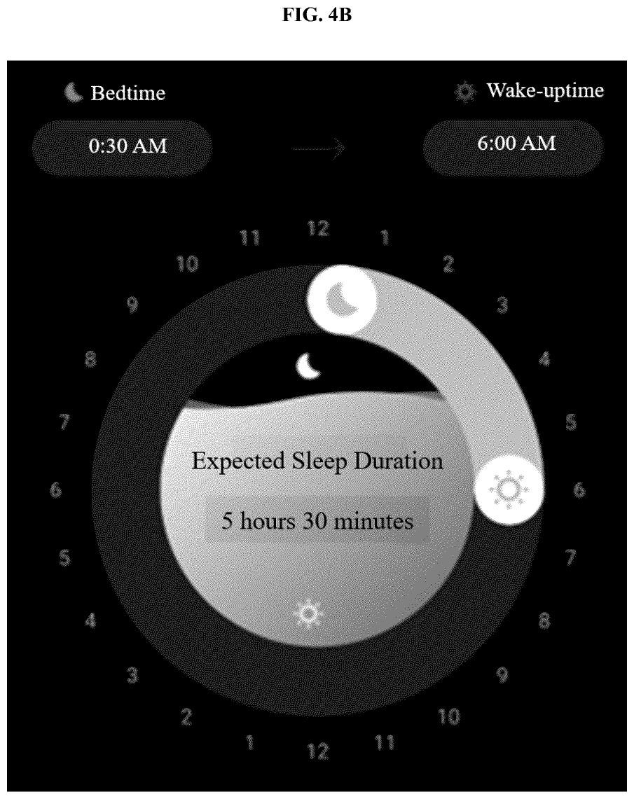

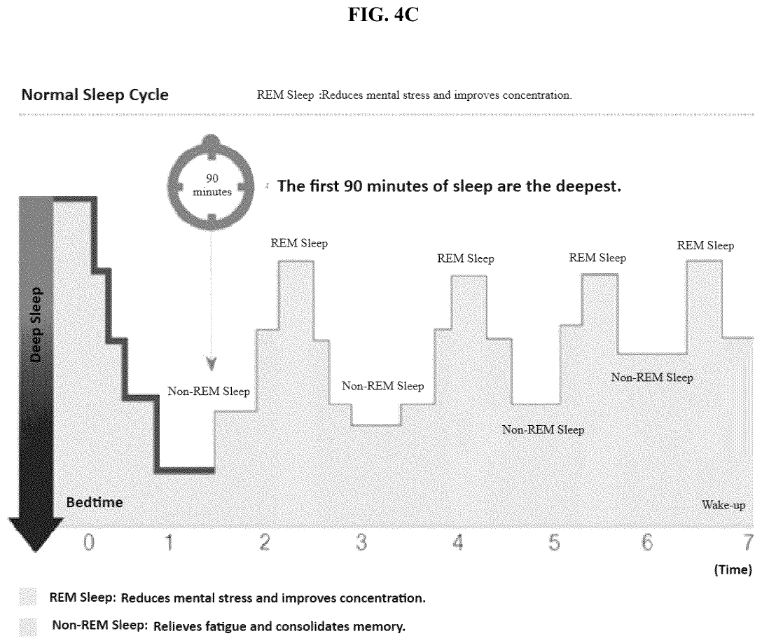

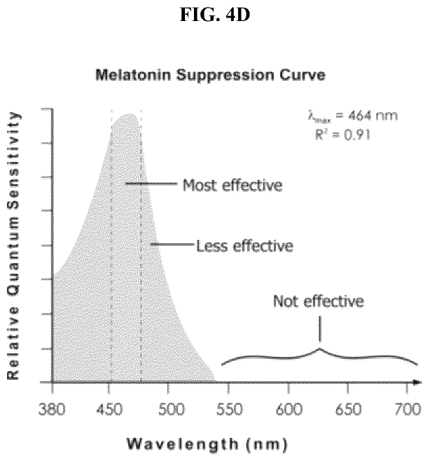

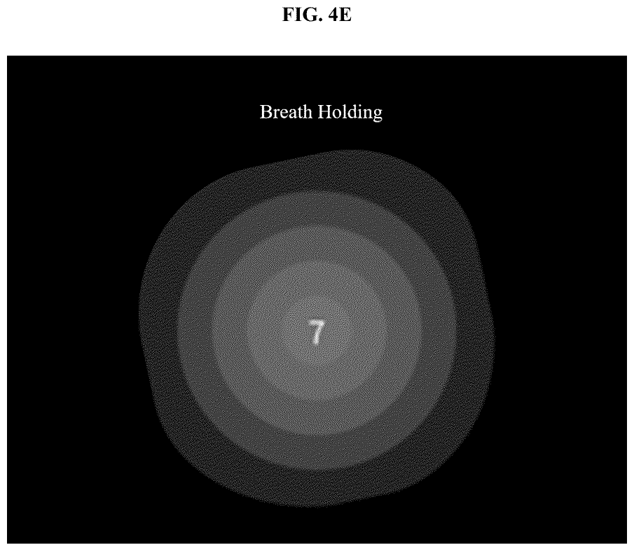

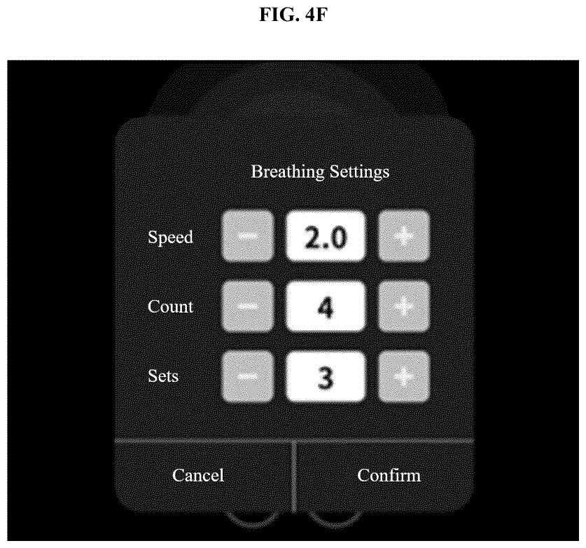

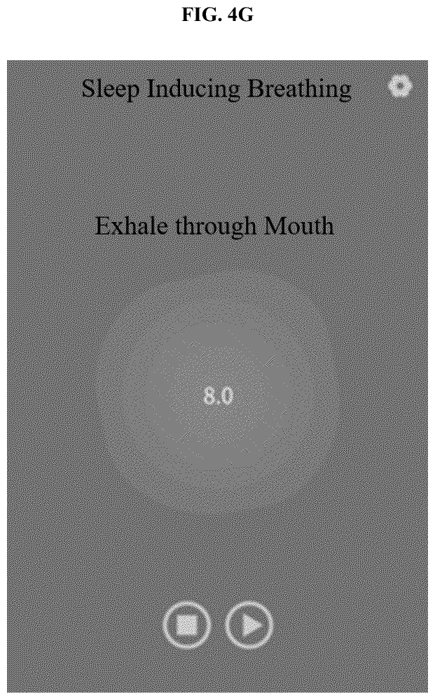

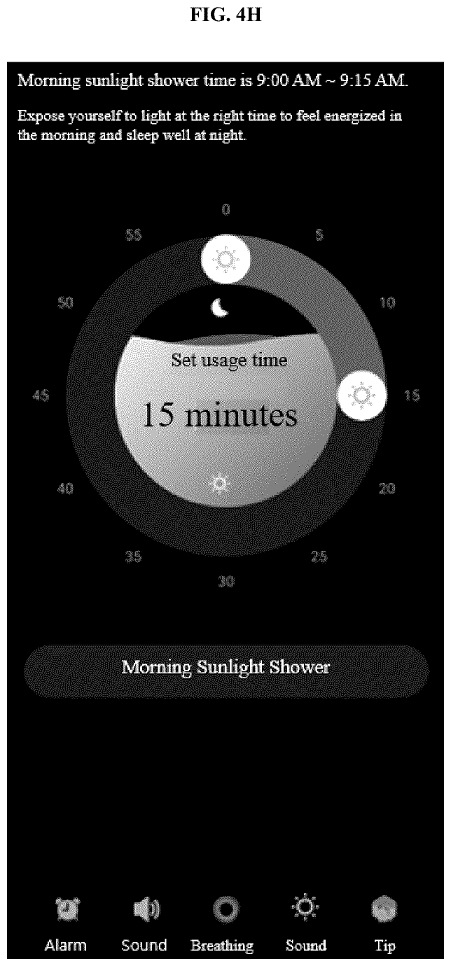

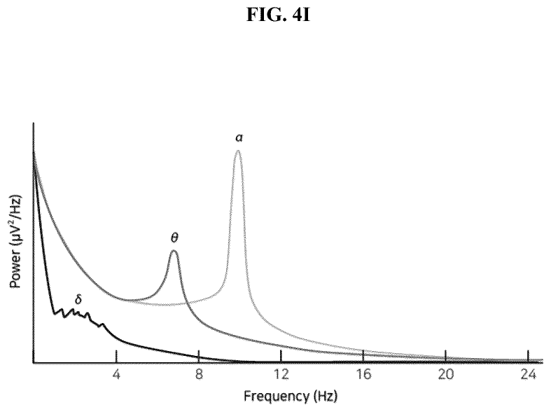

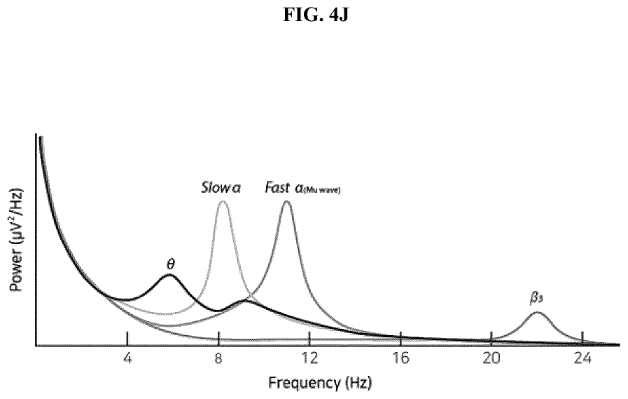

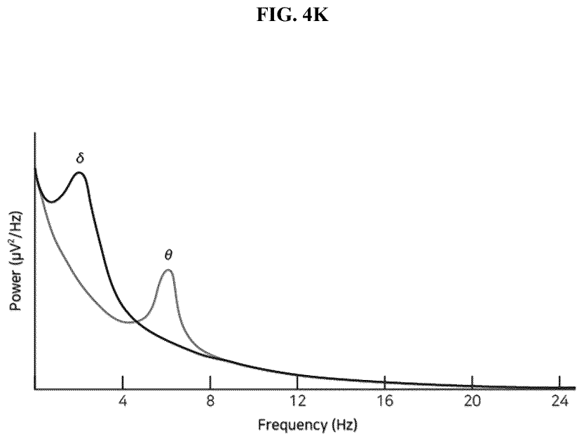

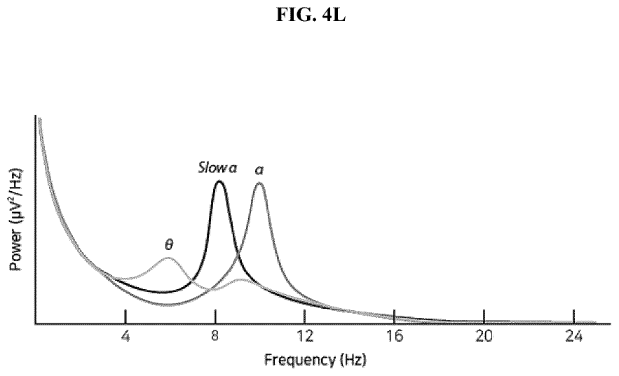

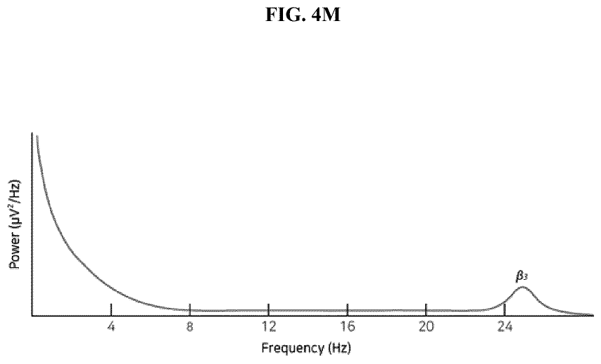

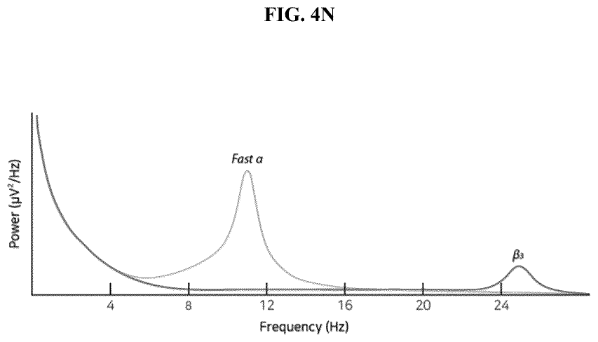

FIG. 2 is a block diagram for explaining a sleep healthcare server included in the system of FIG. 1, and FIGS. 3 and 4 are diagrams for explaining examples in which a sleep healthcare service using brainwave entrainment and autonomic nervous system control according to an embodiment of the present disclosure is implemented.

Referring to FIG. 2, a sleep healthcare server 300 may include a database 310, a setting part 320, a controller 330, a wake-up brainwave induction part 340, a sleep stage coaching part 350, an artificial intelligence part 360, an insomnia alleviation part 370, a sunlight shower part 380, a circadian rhythm alignment part 390, a vagus nerve stimulation part 391, and a bilateral alternating stimulation-in-tactile-form part 393.

When the sleep healthcare server 300 or another server (not shown) operating in conjunction with the sleep healthcare server 300 according to an embodiment of the present disclosure transmits a sleep healthcare service application, program, app page, webpage, or the like using brainwave entrainment and autonomic nervous system control to at least one user terminal 100 and at least one wearable device 400, and at least one information server 500 may install or open the sleep healthcare service application, program, app page, webpage, or the like using brainwave entrainment and autonomic nervous system control. In addition, a service program may be executed on at least one user terminal 100, at least one wearable device 400, and at least one information server 500 using scripts running in a web browser. Here, the web browser is a program that enables the use of web (WWW: World Wide Web) services and displays hypertext described in HTML (Hyper Text Mark-up Language), and examples of the web browser include Chrome, Microsoft Edge, Safari, Firefox, Whale, UC Browser, and the like. Additionally, an application refers to an application on a terminal, and for example, includes an app able to be executed on a mobile terminal (smartphone).

Referring to FIG. 2, the database part 310 may store settings for light, sound, vibration, and a breathing method for brainwave entrainment and autonomic nervous system control at a bedtime and a wake-up time. Brainwaves are biometric information measured through reinforcement interference in the microcurrents of nerve cells when the brain is enhanced. Continuous data is measured at a location with amplitude, frequency, and waveform, which are characteristic of waves. Brainwaves may be used to measure brain activity because the brainwaves exhibit different characteristics depending on the activation state of neural cells. This is why polysomnography uses brainwaves to assess the depth of sleep. During sleep, a person's unconscious reduction in physical activity results in sleep brainwaves exhibiting different characteristics than brainwaves measured during wakefulness (non-sleep). To distinguish sleep stages and determine sleep depth using the characteristics of sleep brainwaves, the sleep stages defined in the Rechtschaffen & Kales Sleep Scoring Manual are employed. These sleep stage criteria are widely used across various research fields, including polysomnography, as shown in Table 1 below.

TABLE 1

| Stage | Frequency Characteristics | Other Characteristics |
|---|---|---|
| Wake | Alpha waves (8-13 Hz) dominant | Noise due to movement and heart |
| | | rate |
| 1 | Mixed waves (2-7 Hz) dominant, alpha | No K-complex and sleep spindle |
| | waves <50% | waves |
| 2 | Slow waves (<2 Hz) <20% | K-complex and sleep spindle waves |
| | | detected |
| 3 | Slow waves 20-50% detected | — |
| 4 | Slow waves >50% detected | — |
| REM | Mixed waves dominant | Noise due to eye movement |

The wake stage is a non-sleep state with alpha waves (8-12 Hz) activated and noise generated due to movement and heartbeat. In stage 1 sleep, mixed brainwaves of 2-7 Hz are measured, and no K-complex and sleep spindle waves are detected. The K-complex refers to a transient, high-amplitude, positively directed waveform, while a sleep spindle is a 12-14 Hz complex wave measured shortly after the K-complex. In Stage 2 sleep, K-complexes and sleep spindles are measured, with slow waveforms below 2 Hz comprising less than 20%. Stage 3 sleep is characterized by slow brainwaves below 2 Hz, measuring between 20% and 50%. Stage 4 sleep includes more than 50% slow brainwaves below 2 Hz. REM sleep is similar to stage 1 sleep but includes noise due to eye movements. Here, the types and frequencies of brainwaves are detailed in Table 2 below.

TABLE 2

| Type of | Frequency | | |
|---|---|---|---|
| Brainwaves | Band | | State of the Brain |
| Delta | 0.5~4 | Hz | Deep sleep state |
| Theta | 4~7 | Hz | Drowsiness, daydreaming, restlessness |
| Alpha | 8~12 | Hz | Relaxed with decreased external focus |
| Sensory Motor | 12~15 | Hz | Maintaining attention in stillness, |
| Rhythm (SMR) | | | intermediate between tension and |
| | | | relaxation |
| Beta | 15~30 | Hz | Active thinking and maintaining focus in |
| | | | an active state |
| Gamma | 31~50 | Hz | Information exchange between cortical |
| | | | and subcortical areas, |
| | | | present in conscious wakefulness and |
| | | | REM sleep, |
| | | | occasionally overlaps with beta waves |

The theory of brainwave entrainment, also known as the Frequency-Following Effect, refers to neural synchronization based on brainwave entrainment through rhythmic external stimuli such as flickering lights, voice, music, and periodic stimuli like music. According to this theory, brainwaves (the brain's large electrical oscillations) synchronize to the desired frequencies induced by these stimuli. This concept has been discussed in publications such as V. A. Korshunov, G. R. Khazankin, and D. S. Ivanishkin, “Development of an Application for Audio-Visual-Tactile Brainwave Entrainment in Patients with Affective and Psychosomatic Disorders,” 2021 IEEE 22nd International Conference of Young Professionals in Electron Devices and Materials (EDM), Souzga, the Altai Republic, Russia, 2021, pp. 551-554, doi: 10.1109/EDM52169.2021.9507617, and H. Norhazman, N. Zaini, M. N. Taib, R. Jailani, and M. F. A. Latip, “Alpha and Beta Sub-waves Patterns when Evoked by External Stressor and Entrained by Binaural Beats Tone,” 2019 IEEE 7th Conference on Systems, Process and Control (ICSPC), Melaka, Malaysia, 2019, pp. 112-117, doi: 10.1109/ICSPC47137.2019.9068008. In one embodiment of the present disclosure, based on the brainwave entrainment theory, light (alpha band and beta band) and sound (monaural beats and binaural beats) are utilized to induce sleep and promote arousal using blue light.

### <Autonomic Nervous System Control>

The sympathetic nervous system originates from the middle part of the spinal cord and distributes signals to various visceral organs, enabling a rapid response to emergency situations. When the sympathetic nervous system is enhanced, the pupils dilate, sweating increases, heart rate accelerates, blood vessels constrict, airways dilate, and gastrointestinal motility decreases. On the other hand, the parasympathetic nervous system originates from the midbrain, pons, and the tail end of the spinal cord, distributing signals to various visceral organs, and stores energy in preparation for emergency situations. When the parasympathetic nervous system is enhanced, the pupils constrict, sweating decreases, heart rate decreases, and some blood vessels may dilate. Furthermore, the airways constrict, and gastrointestinal motility is stimulated. Therefore, the sympathetic nervous system induces arousal, while conversely, the parasympathetic nervous system induces sleep. Thus, red and yellow lights for inducing melatonin production are provided to induce sleep, and a deep breathing method as shown in FIGS. 4E and 4F is induced to enhance the parasympathetic nervous system. Here, the theoretical basis for red and yellow lights inducing melatonin is supported by the paper (Blume C, Garbazza C, Spitschan M. Effects of light on human circadian rhythms, sleep and mood. Somnologie (Berl). 2019 September; 23 (3): 147-156. doi: 10.1007/s11818-019-00215-x. Epub 2019 Aug. 20. PMID: 31534436; PMCID: PMC6751071) and FIG. 4D. Upon awakening, a situation opposite to the above-described method may be induced.

### <Flicker Insertion>

The display of the user terminal 100 may be gradually brightened before the wake-up time, and the display and the flashlight may be turned on at full brightness as the alarm time approaches. In this case, in conjunction with application software, it is possible to insert an image frame into video content from alpha bands or beta bands that flicker—i.e., blink—using LED devices, displays, and flashlights. Flicker may also be generated by adjusting the display refresh rate and blinking the display, flashlight, and associated LEDs. Using flickers, alpha waves or beta waves may be synchronized. This theory is based on the paper mentioned above.

### <Breathing Method>

As shown in FIG. 4E, changes in brain waves may be induced by coaching a user to breathe using shape size changes and numbers through the display of the user terminal 100. This is based on the mechanism discussed in the paper by Kang Seung-wan (2017), “Mechanisms by Which Conscious Breathing Affects the Autonomic Nervous System and Brain Waves,” in Perspectives in Nursing Science, 14(2), 64-69. In this context, the autonomic nervous system, including heart rate, may be regulated through conscious respiratory changes. For example, deep breathing is recommended for individuals who are highly agitated due to intense sympathetic nervous system activation. The idea is to change the heart rate through breathing consciously. The intentional alteration of heart rhythm patterns through breathing may lead to changes in brain waves. At this point, a user-specific interface for controlling breathing may be provided, as illustrated in FIG. 4F. In this interface, a user may select an appropriate breathing method by adjusting the speed, frequency, and sets according to the user's proficiency level while maintaining the ratio of breathing phases. While this breathing method is relatively safe, beginners may experience slight dizziness during initial practice. Normal breathing involves maintaining a balance between inhaling oxygen and exhaling carbon dioxide. Exhaling more than inhaling disrupts this balance, leading to a decrease in carbon dioxide levels in the body. This may constrict blood vessels that supply blood to the brain, potentially leading to symptoms such as dizziness. Therefore, the practice duration may be gradually adjusted, increasing the number of repetitions as the user becomes accustomed. In addition, it is possible to induce closed-eye breathing, and in this case, the display may blink with varying brightness and color, allowing the user to close the eyes and sense light in dark environments while breathing. For example, as shown in FIG. 4E, the light may change from dark black to red, gradually becoming brighter as the user breathes in. Furthermore, it is also possible to configure settings as shown in FIG. 4G: inhale (display transitions from minimum brightness in black to gradually maximum brightness in red)→hold breath (display blinks)→exhale (screen brightness and color gradually dim to minimum brightness)→rest between sets (display brightness and color maintained).

The setting part 320 may receive bedtime and wake-up time settings from the user terminal 100. The user terminal 100 may set a bedtime and a wake-up time. As shown in FIG. 4A, alarms for a bedtime and a wake-up time may be set. In this case, a graphical clock may be provided in either a 12-hour format or a 24-hour format, where a bedtime can be adjusted using the moon icon and a wake-up time can be set using either the sun or alarm clock icon. In addition, as shown in FIG. 4b, it is possible to calculate a difference between a bedtime and a wake-up time to estimate sleep duration. In this setup, an estimated sleep time may be represented within a clock-shaped structure where the filled water gradually approaches the recommended sleep duration, such as 8 hours. The user terminal 100 may be a smartphone and may also be a smartpad, a smartwatch, a PC, a TV, a headset, or the like capable of providing a wake-up alarm using display light and brainwave entrainment.

The controller 330 may output at least one of light, sound, vibration, and a breathing method for brainwave entrainment and autonomic nervous system control, at a bedtime and a wake-up time set by the user terminal 100. In this case, at a bedtime, the user terminal 100 may output a sleep-inducing breathing method for enhancing the parasympathetic nervous system while emitting light with non-melatonin-suppressing wavelengths. At this point, the light with non-melatonin-suppressing wavelengths may include red and yellow lights. In addition, at a wake-up time, the user terminal 100 may insert flickers of the alpha band or beta band for brainwave entrainment and may output blue light to induce arousal. At this point, the blue light may also induce arousal using a blue bandpass filter (Blue BanddPass Filter) that can be attached to LED devices, displays, or flashlights linked to application software. At this point, the theory behind blue light promoting arousal is based on studies such as Liu et al.'s study (Liu D, Li J, Wu J, Dai J, Chen X, Huang Y, Zhang S, Tian B, Mei W. Monochromatic Blue Light Activates Suprachiasmatic Nucleus Neuronal Activity and Promotes Arousal in Mice Under Sevoflurane Anesthesia. Front Neural Circuits. 2020 Aug. 18; 14:55. doi: 10.3389/fncir.2020.00055. PMID: 32973462; PMCID: PMC7461971.) demonstrating blue light activating suprachiasmatic nucleus neuronal activity and promoting arousal in mice under sevoflurane anesthesia, and Figueiro and Leggett's study (Figueiro M G, Leggett S. Intermittent Light Exposures in Humans: A Case for Dual Entrainment in the Treatment of Alzheimer's Disease. Front Neurol. 2021 Mar. 9; 12:625698. doi: 10.3389/fneur.2021.625698. PMID: 33767659; PMCID: PMC7985540.) showing delayed Dim Light Melatonin Onset (DLMO) when exposed to blue light during sleep.

The wake-up brainwave induction part 340 may output sounds of monaural beats or binaural beats for theta, alpha, and beta brainwave entrainment through a speaker of the user terminal 100 before a preset time from the wake-up time. It is possible to induce theta-alpha-beta brainwave patterns sequentially, from theta (low frequency) to alpha and beta (higher frequencies), to gradually awaken a user from sleep and promoting arousal. The theory behind this brainwave entrainment is similar to the above-described brainwave entrainment theory, and thus, a detailed description thereof will be omitted.

The sleep stage coaching part 350 may monitor sleep stages from the wearable device 400 linked to the user terminal 100 from the bedtime, and may output monaural beats or binaural beats set for each sleep stage. At this point, the sleep stage coaching part 350 may output binaural beats when the user terminal 100 is connected to an earphone or headset, and may output monaural beats when the user terminal 100 is connected to a speaker.

The artificial intelligence part 360 may determine sleep stages by querying pre-built AI algorithms with data collected from the wearable device 400. Using the data collected from the wearable device 400, such as GPS, accelerometer, magnetometer, etc., it is possible to determine which stage of sleep the user is currently in based on factors such as the level of movement (tossing and turning) and the current heart rate.

### <Characteristics of Sleep Stages and Biological Signal Sensor>

To accurately discern sleep states, it is essential to analyze the sleep stages within an entire sleep cycle. Generally, the human sleep cycle begins in the wake stage WAKE and predominantly consists of repeated stages of NREM and REM sleep. Physiological signals measured during sleep exhibit distinct characteristics across these stages. During the wake stage WAKE, muscles are activated and breathing and heart rate are irregular. In the NREM stages, compared to the wake stage WAKE, heart rate, breathing, and eye movements slow down. Upon entering the REM stage following NREM, the heart rate and breathing become rapid and irregular again, while muscle tone below the neck decreases.

### <Feature Extraction>

### <Respiratory Rate>

The respiratory rate is subject to noise due to a user's movements. Therefore, at certain points where the respiratory rate is 0 or the value is deemed too high or too low to be considered valid, the data can be corrected by averaging the values before and after. In the REM stage, breathing is characterized by irregularity. Considering the characteristic of increased amplitude due to irregular breathing, the maximum amplitude within a 3-minute window may be measured and used as a feature.

### <Hear Rate>

The heart rate stabilizes upon entering the NREM stage, and during the REM stage, the heart rate graph shows a trend of increasing and decreasing with larger amplitude compared to other sleep stages. Given these characteristics, the amplitude magnitude can be extracted using a 1-minute window.

### <Electrocardiogram (ECG)>

In ECG analysis, the Pan-Tompkins algorithm, which is commonly used, may be applied to extract the QRS complex. Three types of features may be extracted from ECG data: heart rate variability (HRV), amplitude, and the number of R-wave peaks within 1 minute. HRV analysis is based on detecting the R-peak, which is the most prominent feature of the QRS complex. HRV shows different characteristics for each sleep stage and may distinguish between arousal and sleep states with over 87% accuracy. By applying a band-pass filter to the ECG signal, the standard deviation of the R-R peak interval may be calculated at 1-minute intervals using equations 1 and 2, and the short-term heart rate variability element, which is Standard Deviation of NN Interval (Short Term SDNN), may be obtained.

$$MEAN = \sqrt{\frac{{\sum }_{i = 1}^{N} {RRinterval}_{i}}{NumberofRRinterval}} \qquad Equation 1$$

$$ShortTermSDNN = \sqrt{\frac{{\sum }_{i = 1}^{N} {RRinterval}_{i} - MEAN}{NumberofRRinterval}} \qquad Equation 2$$

### <Movement>

Data collected from the 3-axis accelerometer is used to determine movement during sleep. Since directionality is not considered, the 3-dimensional accelerometer values may be reduced to a 1-dimensional value and converted into an intensity value that represents the intensity of movement during sleep. By substituting the acceleration at time n along the XYZ axes into Equation 3, the movement intensity In at time n may be obtained.

$${I}_{n} = \sqrt{{X}^{2} n + {Y}^{2} n + {Z}^{2} n} \qquad Equation 3$$

### <Sleep Cycle>

Most living things have a 24-hour circadian rhythm. To assign a Clock Proxy value representing the sleep cycle instead of an absolute time corresponding to a measured biosignal, an existing biological clock modeling technique may be used to allocate values from the start to the end of the measurement period. The X-axis represents the sleep time extracted at 1-second intervals, and the Y-axis is assigned the value of the half-cycle sine graph corresponding to the X value.

### <Data Generation, Learning, and Classification>

Raw data collected from each sensor is visualized to interpret the waveforms appearing in each sleep stage. Then, by comparing with the waveforms observed in previous studies, labels may be assigned as follows: WAKE state as 0, NREM as 1, and REM as 2. After preprocessing using Python Pandas and Numpy libraries, the data may be integrated into a single data frame based on a measurement time and saved as a CSV file. This CSV file may include features such as heart rate variability, respiratory variability, movement intensity, and Clock Proxy. The preprocessed data may then be used as training and test data for an SVM classifier.

The SVM classifier finds the decision hyperplane that separates each class and maximizes a distance to the nearest samples (Support Vectors), demonstrating excellent generalization ability in classification. Therefore, erroneous data has less impact, and occurrences of overfitting are reduced. By using the Python Scikit-Learn library, sleep data that cannot be linearly separated may be classified by mapping the data to a higher dimension than the finite dimension of the initial problem using the RBF (Radial Basis Function) kernel. To enhance the performance of sleep stage classification, a One-versus-One (OvO) method is chosen over a One-versus-Rest (OvR) method to improve learning accuracy, despite a longer processing time. The parameters C and σ of the RBF kernel may be empirically set to 1 and 0.1, respectively, using Grid Search.

Based on the results of classifying sleep stages using the SVM classifier, the ratio of each sleep stage to total sleep and the sleep efficiency, which is the ratio of actual sleep time to recorded time, may be calculated. To provide a user with the changes in sleep stages over the entire sleep period and the ratio of each sleep stage, the Python Matplotlib library may be used to visualize the sleep stages over the entire sleep period as a hypnogram and the distribution of each sleep stage as a pie chart. Through a hypnogram, sleep stages may be identified, enabling preparation for sleep and wakefulness according to the sleep stages.

If insomnia is registered or is not registered on the user terminal 100, the insomnia alleviation part 370 may provide monaural or binaural beats to synchronize alpha, theta, and delta waves and stop brainwave entrainment after a deep sleep stage to induce REM sleep. If hypersomnia is registered or is not registered on the user terminal 100, the insomnia alleviation part 370 may provide monaural or binaural beats to synchronize alpha, theta, and delta waves in sequence manually. Here, since there may be occasional cases of inability to sleep even if insomnia is registered, a case where insomnia is not registered implies inducing brainwave entrainment manually or automatically (based on sleep stages) in such instances. Similarly, since there may be occasional cases of excessive sleep even if hypersomnia is not registered, a case where hypersomnia is not registered implies inducing brainwave entrainment manually or automatically (based on sleep stages) in such instances.

The theoretical basis for synchronizing alpha, theta, and delta waves in cases of insomnia and theta, alpha, and beta waves in cases of hypersomnia is grounded in a paper (Gantt M A. Study protocol to support the development of an all-night binaural beat frequency audio program to entrain sleep. Front Neurol. 2023 Jan. 26; 14:1024726. doi: 10.3389/fneur.2023.1024726. PMID: 36779067; PMCID: PMC9909225). When the user terminal 100 is connected to an earphone or headset, binaural beats may be output, and when the user terminal 100 is connected to a speaker, monaural beats may be output.

In this case, as people age, total sleep time, slow-wave sleep, and sleep efficiency may decrease, while wakefulness after sleep onset and sleep latency may increase, and therefore, age should be considered. Additionally, since women generally maintain slow-wave sleep longer than men, gender may also be considered. When insomnia or hypersomnia is alleviated, it may help treat circadian rhythm disorders, bipolar disorder, depression, manic-depressive disorder, anxiety disorder, Alzheimer's disease, and Parkinson's disease and may assist in readjusting circadian rhythms for travelers and shift workers adapting to time zone changes.

### <90-Minute Golden Time Sleep Rule>

According to Dr. Seiji Nishino, director of the Sleep and Circadian Neurobiology Research Institute at Stanford University, as introduced in the best-selling book “Stanford High-Efficiency Sleep Method,” the first sleep cycle immediately after falling asleep significantly influences the overall sleep quality, as shown in FIG. 4C. Within the first 70 to 90 minutes of the first sleep cycle, a non-REM sleep stage is observed. The non-REM sleep is a deep sleep stage that replenishes fatigue and consolidates memory. The 90 minutes during which the first non-REM sleep occurs is called the “golden time of sleep.” Sleeping deeply during the first 90 minutes after falling asleep may provide a refreshing feeling the next day, even with less total sleep time than usual. For modern individuals who inevitably have limited sleep time, achieving deep and stable sleep during the first cycle may be the best option. As wakefulness during the day increases, the desire to sleep also intensifies. This desire is called sleep pressure. The sleep pressure peaks during the first 90 minutes after falling asleep, that is, during the golden time of sleep. Sleeping deeply during the golden time of sleep significantly reduces sleep pressure, eases the urge to sleep, and diminishes fatigue. The golden time of sleep regulates the autonomic nervous system through sleep, promotes growth hormone secretion, and enhances brain function.

Patients with depression often experience insufficient early non-REM sleep, leading to premature onset of REM sleep, and thus, waking up during REM sleep is used as a way for treatment of depression. According to the paper by Palagini L, Baglioni C, Ciapparelli A, Gemignani A, Riemann D, “REM sleep dysregulation in depression: state of the art,” there is a strong association between REM sleep disorders and depression. This appears to increase the risk of onset and recurrence of depression, and even impact treatment response, regardless of the method used for treating depression. The key role of REM sleep is to regulate emotional reactivity and emotional information processing in depression. Neuro-metabolic changes in depression are induced or exacerbated by excessive activation of REM sleep. Accordingly, in one embodiment of the present disclosure, a deep sleep stage time in the first sleep cycle may be increased using delta wave synchronization. Monitoring sleep stages and waking up during the REM sleep stage may deactivate REM sleep overactivity.

### <Binaural Beats in the Inaudible Frequency Range>

Brain waves may also be synchronized through monaural beats or binaural beats using inaudible frequencies. This is based on a study (Choi M H, Jung J J, Kim K B, Kim Y J, Lee J H, Kim H S, Yi J H, Kang O R, Kang Y T, Chung S C. Effect of binaural beat in the inaudible band on EEG (STROBE). Medicine (Baltimore). 2022 Jul. 1; 101 (26): e29819. doi: 10.1097/M D.0000000000029819. PMID: 35777013; PMCID: PMC9239629.) that suggests that binaural beats with inaudible frequencies can induce specific brainwaves similar to the brainwave-inducing effects of binaural beats at typical audible frequencies.

The sunlight shower part 380 can provide blue light at a wakeup time. The alarm and light therapy time may be provided as shown in FIG. 4H. Using user data, an algorithm may predict effective usage times and amounts of sunlight shower, and as the recommended usage time and amount approach, the clock shape may be filled. An optimized morning sunlight shower time may be determined considering a user's sleep pattern, indicating when the user falls asleep and wakes up. Alternatively, when to take a morning sun shower may be determined based on when the user needs to sleep and wake up. The best approach for conditions such as insomnia or oversleeping is to establish a fixed wake-up time, and to expose oneself to sunlight immediately upon waking. Direct exposure to the morning sunlight, rich in blue light, suppresses melatonin, which is a sleep hormone, and promotes the secretion of serotonin which is converted to melatonin after 14 to 15 hours to induce deep sleep. A guide and alert for sunlight shower or light therapy about 14 to 15 hours before a predicted bedtime or sleep time may be provided.

The circadian rhythm alignment part 390 may cause blue light to be emitted based on a circadian rhythm determined by user data of the user terminal 100 in addition to the wake-up time. At this point, sleep data, light exposure data, and activity data may be measured, including sleep stages, sleep duration, a bedtime, a wake-up time, light exposure from light sensors, light exposure, GPS data, and heart rate data. In addition, the circadian rhythm may be measured using a preset algorithm, circadian rhythm disorders may be diagnosed, and a personalized light therapy may be provided through circadian rhythm data analysis. That is, the timing, intensity, and duration of blue light exposure may be adjusted. Since the human circadian clock runs on a cycle that is slightly longer than 24 hours, the circadian clock may be advanced daily using brainwave entrainment.

If a circadian rhythm disorder is monitored, the vagus nerve stimulation part 391 may output vibrations corresponding to a non-invasive vagus nerve stimulation device on the user terminal 100 or may connect the user terminal 100 to the vagus nerve stimulation device to operate the vagus nerve stimulation device. The non-invasive vagus nerve stimulation device is a portable user-friendly device (gammaCore™) that delivers high-frequency electrical pulses (25 Hz, 2 minutes). The non-invasive vagus nerve stimulation the afferent fibers of the vagus nerve in the skin of the neck, particularly controlling the central inflow of the vagus nerve into the trigeminal vascular system. The efficacy of nVNS for cluster headaches was evaluated in two double-blind placebo-controlled trials. The Headache (ACT-1) study treated 133 patients, using nVNS to manage five cluster headache attacks. There was a trend towards more frequent pain reduction or disappearance within 15 minutes of treatment in the entire cluster headache patient group that was treated compared to the placebo group at the first treatment (26.7% vs. 15.1%, p=0.1). Additionally, a statistically significant difference was reported, particularly in patients with episodic cluster headaches (34.2% vs. 10.6%, p=0.008).” The ACT-2 study also demonstrated that nVNS treatment was more effective than placebo in the episodic cluster headache group, showing a higher proportion of headache attacks achieving pain disappearance within 15 minutes (48% vs. 6%, p<0.01). Thus, demonstrating moderate to superior efficacy and good tolerability in cluster headache patients, nVNS may be considered for acute treatment in the absence of standard therapy or insufficient relief from medications. The theoretical basis for this is supported by the paper (Silberstein S D, Mechtler L L, Kudrow D B, Calhoun A H, McClure C, Saper J R, Liebler E J, Rubenstein Engel E, Tepper S J; ACTI Study Group. Non-Invasive Vagus Nerve Stimulation for the Acute Treatment of Cluster Headache: Findings from the Randomized, Double-Blind, Sham-Controlled ACTI Study. Headache. 2016 Scp; 56 (8): 1317-32. doi: 10.1111/head.12896. PMID: 27593728; PMCID: PMC5113831.).

This may also be achieved using Sensate®Somacoustics, which is based on the paper “Sensate® Somacoustics: A New Wave for Stress Management. Volume I By, Scott McDoniel, Ph.D., M.Ed. 1 & Stefan Chmelik, M.Sc. 2 1. Faculty; College of Health Professions, Walden University, Minneapolis, MN 2. BioSelf Technologies, Ltd, London, UK.” The U.S. FDA has approved a vagus nerve stimulation device as long-term adjunctive therapy for chronic recurrent depression in patients over 18 years of age. In this case, whole-body vibrations between 20 and 30 Hz significantly increase brain secretion levels and increase the necessary protein for neural plasticity. Here, Sensate® is a non-electrical vibration device for stress management, which improves parasympathetic nervous system (PNS) responses to chronic stress using low-frequency technology (<50 Hz). The two theoretical concepts of the Sensate® device are bone conduction and thoracic resonance. In the rib cage, the amplification may significantly increase due to bone movement, and additional energy may result from resonance characteristics within the chest. Low-frequency sound waves (60 Hz) travel longer distances around internal organs, and the afferent nerve branches connected to the vagus nerve are located throughout the chest cavity. Therefore, the Sensate® device is a new method of vagus nerve stimulation that enhances parasympathetic nervous system functions. When the parasympathetic nervous system is enhanced, symptoms such as anxiety, insomnia, and other stress-related symptoms may improve.

When a stress level exceeding a predetermined stress threshold is monitored or manually set by the wearable device 400 linked to the user terminal 400, the bilateral alternating stimulation-in-tactile-form part 393 may output vibrations of a preset frequency while the user terminal 100 is placed on one hand or wrist of the user and the wearable device 400 on the other hand or wrist of the user. At this point, the frequency, intensity, duration, and number of vibrations may be set to increase or decrease from the user terminal 100. For example, a TouchPoint provided by the Wearable Stress Relief Device TouchPoints-TheTouchPoint Solution™ (https://thetouchpointsolution.com/) may be used, which is a wristband that reduces stress by gently alternating vibrations set in both wrists. According to a paper (Leal-Junior, Ernesto Cesar Pinto et al. “A Triple-Blind, Placebo-Controlled Randomized Trial of the Effect of Bilateral Alternating Somatosensory Stimulation on Reducing Stress-Related Cortisol and Anxiety During and After the Trier Social Stress Test.” Journal of Biotechnology and Biomedical Science (2019): n. pag.), bilateral alternating stimulation (Bilateral alternating Stimulationin Tactile form) may provide non-invasive and non-pharmacological means to manage stress. In other words, bilateral alternating somatosensory stimulation may be effective in reducing subjective stress and anxiety levels and may be beneficial for patients with anxiety.

Moreover, this paper by Serin et al. (Amy Serin, Nathan S. Hageman, Emily Kade (2018) The Therapeutic Effect of Bilateral Alternating Stimulation Tactile Form Technology on the Stress Response. Journal of Biotechnology and Biomedical Science-1 (2): 42-47. https://doi.org/10.14302/issn.2576-6694.jbbs-18-1887) also states that it provides stress reduction effects for individuals with high anxiety levels, such as post-traumatic stress disorder (PTSD), by regulating the electrical activity of brain networks that mediate stress responses In addition, the results of the study using BLAST are consistent with the hypothesis of alternating hemisphere activation, which suggests that rapid alternation of electrical activity patterns in the two hemispheres can increase interactions between brain hemispheres. According to Harper's EEG study on PTSD subjects, BLAST was found to have a depolarizing effect on synapses in the amygdala activated during fear-based memory recall. This result suggests that BLAST may impact the electrical activity of key brain areas associated with stress and anxiety, potentially alleviating human stress responses and reducing sensory perception associated with painful memories or physical pain. By reducing electrical activity in key areas of the Salience network, BLAST may reduce sympathetic nervous system activation. In addition, individuals with obsessive-compulsive disorder may experience significant anxiety due to limited attention caused by internal and external distractions, leading to heightened vigilance. Therefore, using BLAST leads to a substantial reduction in excessive activity.

### <Applicable Fields>

A study conducted by Roh et al. (Roh H W, Choi J G, Kim N R, Choe Y S, Choi J W, Cho S M, Seo S W, Park B, Hong C H, Yoon D, Son S J, Kim E Y. Associations of rest-activity patterns with amyloid burden, medial temporal lobe atrophy, and cognitive impairment. EBioMedicine. 2020 August; 58:102881. doi: 10.1016/j.ebiom.2020.102881. Epub 2020 Jul. 28. PMID: 32736306; PMCID: PMC7394758.) reported that patients with Alzheimer's disease show a delay in entering deep sleep by about an hour compared to patients with non-Alzheimer's cognitive impairment. In another paper by Kim et al. (Kim J, Park I, Jang S, Choi M, Kim D, Sun W, Choe Y, Choi J W, Moon C, Park S H, Choe H K, Kim K. Pharmacological Rescue with SR8278, a Circadian Nuclear Receptor REV-ERBa Antagonist as a Therapy for Mood Disorders in Parkinson's Disease. Neurotherapeutics. 2022 March; 19 (2): 592-607. doi: 10.1007/s13311-022-01215-w. Epub 2022 Mar. 23. Erratumin: Neurotherapeutics. 2022 May 2: PMID: 35322351; PMCID: PMC9226214.), Parkinson's disease patients experience not only motor disorders but also sleep disturbances and circadian rhythm disorders, among which evening symptoms such as anxiety and depression are termed sundowning syndrome. Furthermore, the paper by Lee et al. (2018), “Is Circadian Rhythm Dysregulation a Core Mechanism Underlying Bipolar Disorder?”, states that dysregulation of circadian rhythms is closely associated with bipolar disorder, and managing circadian rhythms significantly improves the prognosis of bipolar disorder and that effective treatment methods need to be developed for this purpose. Additionally, according to iMediCine, if brainwave entrainment can normalize brainwaves compared to those of healthy individuals, it is possible to alleviate the aforementioned conditions and disorders such as mild cognitive impairment (FIG. 4I), ADHD (FIG. 4J), schizophrenia (FIG. 4K), depression (FIG. 4I), chronic pain (FIG. 4M), and anxiety disorders (FIG. 4N). Therefore, using brainwave entrainment for mild cognitive impairment, Alzheimer's disease, ADHD, schizophrenia, depression, and chronic pain may alleviate these symptoms or conditions.

TABLE 3

| | | | | Autonomic | |
|---|---|---|---|---|---|
| | | | | Nervous | |
| Disease | Light | Sound | Breathing | System | Brain waves |
| Mild | Gamma Band/ | Monoaural | Deep | Enhancement | Alpha Wave |
| Cognitive | Alpha Band | Beat/Binaural | Breathing | | |
| Impairment | Flicker | Beat | | | |
| ADHD/ | Blue Light/ | Monoaural | | | Beta Wave |
| Schizophrenia/ | Beta Band | Beat/Binaural | | | |
| OCD | Flicker | Beat | | | |
| Enhanced | Blue Light/ | Monoaural | | | Beta Wave |
| Learning | Beta Band or | Beat/Binaural | | | (Concentration) |
| | Gamma Band | Beat | | | Gamma Wave |
| | Flicker | | | | (Memory) |
| Depression | Blue light | Monoaural | Deep | Enhancement | Alpha Wave |
| | | Beat (Right | Breathing | | Beta Wave |
| | | Brain/Left Ear) | | | |
| Chronic Pain | Blue Light/ | Monoaural | Deep | Enhancement | Alpha Wave |
| | Alpha Band | Beat/Binaural | Breathing | | |
| | Flicker | Beat | | | |
| Hypertension | Alpha Band | Monoaural | Deep | Enhancement | Alpha Wave |
| | Flicker | Beat/Binaural | Breathing | | |
| | | Beat | | | |

In the case of depression, brainwave entrainment may be implemented by activating beta waves, balancing the frequency between the left and right hemispheres of the brain. The goal is to decrease alpha waves in the left hemisphere and increase alpha waves in the right hemisphere to achieve equilibrium. To this end, monaural beats for alpha wave synchronization in the right hemisphere are delivered through the left ear. During a blue light therapy, monaural or binaural beats for beta wave synchronization are played. Coaching deep breathing enhances the parasympathetic nervous system and strengthens alpha brain waves. Alternatively, monaural or binaural beats of the gamma band may be played to induce gamma brain waves. This is because while the exact cause of depression is not well known, a recent paper by Li et al. (Li Q, Takeuchi Y, Wang J, Gellert L, Barcsai L, Pedraza L K, Nagy A J, Kozak G, Nakai S, Kato S, Kobayashi K, Ohsawa M, Horvath G, Kekesi G, Lrincz M L, Devinsky O, Buzsaki G, Berenyi A. Reinstating olfactory bulb-derived limbic gamma oscillations alleviates depression-like behavioral deficits in rodents. Neuron. 2023 Jul. 5; 111 (13): 2065-2075.e5. doi: 10.1016/j.neuron.2023.04.013. Epub 2023 May 9. PMID: 37164008; PMCID: PMC10321244.) suggests a decrease in gamma wave oscillations as a new biomarker. Here, the quantitative electroencephalogram (QEEG) of FIG. 4I through FIG. 4N is analyzed as shown in Table 4 below. The source of FIGS. 4I to 4N is iMediSync, as described above. By implementing synchronization to fill excessive or insufficient brain waves in this manner, each condition or symptom may be alleviated.

TABLE 4

| | | |
|---|---|---|
| FIG. | Mild | In brainwaves of normal elderly people, normal brain waves |
| 4I | Cognitive | such as alpha waves are observed (yellow). |
| | Impairment | In brainwaves of patients with mild cognitive impairment, slow |
| | | waves such as theta waves are observed (green). |
| | | In brainwaves of Alzheimer's patients, very slow waves such as |
| | | delta waves and theta waves are observed (blue). |
| FIG. | ADHD | Pattern of excessive alpha waves + normal alpha frequency in the |
| 4J | | frontal lobe (gray) |
| | | Pattern of excessive alpha waves + slower alpha frequency in |
| | | the frontal lobe (yellow) |
| | | Pattern of excessive theta waves in the frontal lobe (blue) |
| | | Pattern of excessive beta 3 waves pattern (green) |
| FIG. | Schizophrenia | Pattern of excessive delta waves in the frontal lobe (blue) |
| 4K | | Pattern of excessive theta waves in the frontal lobe (green) * |
| | | This may co-occur with alpha wave deficiency. |
| FIG. | Depression | Pattern of excessive alpha waves in the frontal lobe (green) |
| 4l | | Pattern of slower alpha waves (blue) |
| | | Pattern of excessive theta waves in the frontal lobe (yellow) |
| FIG. | Chronic Pain | Pattern of excessive beta 3 waves in the parietal lobe (green) |
| 4M | | |
| FIG. | Anxiety | Pattern of excessive beta 3 waves in the central lobe (green) |
| 4N | disorder | Pattern of faster alpha waves(yellow) |

Hereinafter, an operation process based on the configuration of the above-described sleep healthcare server of FIG. 2 will be described in detail with reference to FIGS. 3 and 4. However, what is described below is merely an example, and embodiments of the disclosure are not limited thereto.

Referring to FIG. 3, when a sleep healthcare server 300 links and stores the user terminal 100 and the wearable device 400 and sets the bedtime and wake-up time in the user terminal 100, as shown in (a), it is possible to induce sleep at the bedtime and induce arousal at the wake-up time through brain entrainment and autonomic nerve system control, as shown in (b). In addition, it is also possible to lower a stress level through bilateral alternating stimulation in tactile form, as shown in (c), and to alleviate chronic pain through microstimulation, as shown in (d). Other diseases whose symptoms can be alleviated through various brainwave entrainments are listed in Table 3, and redundant descriptions thereof will be omitted. In addition, FIG. 4 has been described in FIG. 2, and redundant descriptions thereof will be omitted.

Any details not explained regarding the method for providing a sleep healthcare service using brainwave entrainment and autonomic nervous system control, as illustrated in FIGS. 2 through 4, may be identical to those explained through FIG. 1 or may be easily inferred from the described content. Therefore, detailed descriptions thereof will be omitted.

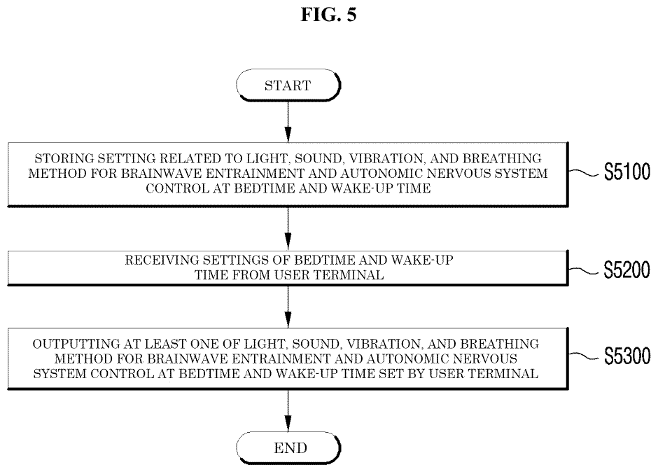

FIG. 5 is a diagram illustrating a process in which data is transmitted and received between components included in the sleep healthcare service providing system using brainwave entrainment and autonomic nervous system control of FIG. 1 according to an embodiment of the present disclosure. Hereafter, an example of the process of data transmission and reception among the various components will be explained with reference to FIG. 5. However, it should be understood that the present disclosure is not limited to this embodiment, and it is evident to those skilled in the art that the process of data transmission and reception shown in FIG. 5 may vary according to the various embodiments previously described.

Referring to FIG. 5, a sleep healthcare server may store settings for light, sound, vibration, and breathing methods for brainwave entrainment and autonomic nervous system control at a bedtime and a wake-up time.

In addition, the sleep healthcare server may receive bedtime and wake-up time settings from a user terminal in operation S5200, and may output at least one of light, sound, vibration, and a breathing method for brainwave entrainment and autonomic nervous system control at a bedtime and a wake-up time set by the user terminal in operation S5300.

The sequence between the above-described operations S5100 to S5300 is merely an example and is not limited thereto. That is, the sequence between the above-described operations S5100 to S5300 may change, and some of the operations may be executed simultaneously or omitted.

Matters that have not been explained about the method of providing a sleep healthcare service using brainwave entrainment and autonomic nervous system control in FIG. 5 5 may be the same as those described with reference to FIGS. 1 to 4 or may be easily inferred from the descriptions of FIGS. 1 to 4. Therefore, those descriptions will be omitted.

The method of providing a sleep healthcare service using brainwave entrainment and autonomic nervous system control according to an embodiment described with reference to FIG. 5 may be implemented in the form of a recording medium containing computer-executable instructions or commands, such as an application or program module executable on a computer. The computer-readable medium may be an available medium that is accessible by a computer. The computer-readable storage medium may include a volatile medium, a non-volatile medium, a separable medium, and/or an inseparable medium. In addition, the computer-readable medium may include a computer storage medium. The computer storage medium may include a volatile medium, a non-volatile medium, a separable medium, and/or an inseparable medium that is implemented in any method or scheme to store computer-readable commands, data architecture, program modules, or other data or information.

The method of providing a sleep healthcare service using brainwave entrainment and autonomic nervous system control according to an embodiment of the present disclosure may be executed by an application installed on a terminal, including a platform equipped in the terminal or a program included in the operating system of the terminal), or may be executed by an application (or program) installed by the user on a master terminal via an application provider server, such as a web server, associated with the service or method, an application, or an application store server. According to an embodiment, the above-described method of providing a sleep healthcare service using brainwave entrainment and autonomic nervous system control according to an embodiment of the present disclosure may be implemented in an application or program installed as default on the terminal or installed directly by the user and may be recorded in a recording medium or storage medium readable by a terminal or computer.

Although embodiments of the present disclosure have been described with reference to the accompanying drawings, it will be appreciated by one of ordinary skill in the art that the present disclosure may be implemented in other various specific forms without changing the essence or technical spirit of the present disclosure. Thus, it should be noted that the above-described embodiments are provided as examples and should not be interpreted as limiting. Each of the components may be separated into two or more units or modules to perform its function(s) or operation(s), and two or more of the components may be integrated into a single unit or module to perform their functions or operations.

It should be noted that the scope of the present disclosure is defined by the appended claims rather than the described description of the embodiments and include all modifications or changes made to the claims or equivalents of the claims.

What is claimed is:

1. A system for providing a sleep healthcare service using brainwave entrainment and autonomous nervous system control, the system comprising:
a user terminal configured to set a bedtime and a wake-up time, at the bedtime, output a sleep-inducing breathing method for enhancing the parasympathetic nervous system, while emitting light of non-melatonin-suppressing wavelengths, and at the wake-up time, insert and output an Alpha-Band or Beta-Band flicker for brainwave synchronization of alpha or beta waves, and output blue light to induce arousal; and
a sleep healthcare server comprising a database part for storing settings related to light, sound, vibration, and a breathing method for brainwave entrainment and autonomic nervous system control at the bedtime and the wake-up time, a setting part for receiving bedtime and the wake-up time settings from the user terminal, and a controller for outputting at least one of the light, sound, vibration, and breathing method for brainwave entrainment and autonomic nervous system control at the bedtime and the wake-up time set by the user terminal.

2. The system of claim 1, wherein the light of the non-melatonin-suppressing wavelengths comprises red and yellow lights.

3. The system of claim 1, wherein the sleep healthcare server further comprises a wake-up brainwave induction part configured to output the sound of monaural beats or binaural beats through a speaker, earphones, or a headset of the user terminal for brainwave entrainment of theta, alpha, and beta waves, starting at a preset time before the wake-up time.

4. The system of claim 1,
wherein the sleep healthcare server further comprises a sleep stage coaching part configured to monitor sleep stages through a wearable device linked to the user terminal, starting from the bedtime, and output preset monaural beats or binaural beats at audible or inaudible frequencies for each of the sleep stages, and
wherein the binaural beats are output when the user terminal is connected to the earphone or headset, and the monaural beats are output when the user terminal is connected to the speaker.

5. The system of claim 4, wherein the sleep healthcare server further comprises an artificial intelligence part configured to identify the sleep stages by inputting data collected from the user terminal or a wearable device linked to the user terminal into a pre-established AI algorithm as a query.

6. The system of claim 4,
wherein the sleep healthcare server further comprises an insomnia alleviation part configured to, in response to the user terminal being registered with or without insomnia, provide monaural beats or binaural beats to entrain alpha, theta, and delta waves manually according to sleep stages and stop brainwave entrainment after a deep sleep stage to induce REM sleep, and in response to the user terminal being registered with or without hypersomnia, provide the monaural beats or binaural beats to entrain theta, alpha, and beta waves in sequence manually, and
wherein the binaural beats are output when the user terminal is connected to the earphone or headset, and the monaural beats are output when the user terminal is connected to the speaker.

7. The system of claim 1, wherein the sleep healthcare server further comprises a sunlight shower part configured to provide a blue light at the wake-up time.

8. The system of claim 7, wherein the sleep healthcare server further comprises a circadian rhythm alignment part configured to schedule the emission of the blue light based on a circadian rhythm identified from user data of the user terminal, in addition to the wake-up time.

9. The system of claim 8, wherein the sleep healthcare server further comprises a vagus nerve stimulation part configured to, when a circadian rhythm disorder is monitored, output vibrations corresponding to a non-invasive vagus nerve stimulation (nVNS) device on the user terminal or connect the user terminal to the vagus nerve stimulation device to operate the vagus nerve stimulation device.

10. The system of claim 1,
wherein the sleep healthcare server further comprises a bilateral alternating stimulation-in-tactile-form part configured to, when a stress level exceeding a preset stress threshold is monitored or manually set by a wearable device linked to the user terminal, output vibrations of a preset frequency with the wearable device being placed on one hand or wrist of the user or on the other hand or wrist of the user, and
wherein a frequency, an intensity, a duration, and a number of the vibrations are set to increase or decrease on the user terminal.
